(19) United States
(12) Patent Application Publication
(10) Pub. No.: US 2009/0076550 A1
(43) Pub. Date: Mar. 19, 2009

(54) SPINAL FIXATION SYSTEM CONNECTORS

(75) Inventors: Andrew E. Bernhardt, JR., Seymour, CT (US); Kevin Durst, Salt Lake City, UT (US); Nathan Otis Nelson, South Jordan, UT (US); Bryan Joseph Martin, Cedar Hills, UT (US)

Correspondence Address: STOEL RIVES LLP - SLC, 201 SOUTH MAIN STREET, SUITE 1100, ONE UTAH CENTER, SALT LAKE CITY, UT, 84111, US

(73) Assignee: ORTHO DEVELOPMENT CORPORATION, Draper, UT (US)

(21) Appl. No.: 11/857,124

(22) Filed: Sep. 18, 2007

(51) Int. Cl. A61B 17/70; A61B 17/56; A61B 17/04; A61B 17/58

(52) U.S. Cl. 606/246; 606/278; 606/301; 606/305; 606/308

(57) ABSTRACT

Disclosed are embodiments of spinal fixation system connectors and related methods and apparatus. In one embodiment, a connector body is provided which comprises a slot configured to allow a spinal fixation rod to extend therethrough. The connector may also include a cap configured to be connected with the connector body and thereby circumscribe a portion of the rod. The cap may comprise at least two interior groove portions that may be configured to receive top portions of two opposing sidewalls on the connector body when the cap is engaged with the connector body.

### BRIEF DESCRIPTION OF THE DRAWINGS

Understanding that drawings depict only certain preferred embodiments of the invention and are therefore not to be considered limiting of its scope, the preferred embodiments will be described and explained with additional specificity and detail through the use of the accompanying drawings in which:

FIG. 1 is an exploded perspective view of several components of one embodiment of a spinal fixation system.

FIG. 2 is a front elevation view of the assembled components of FIG. 1 but without the connector body cap.

FIG. 3 is an exploded top perspective view of one embodiment of a connector body cap.

FIG. 4 is a bottom perspective view of the connector body cap of FIG. 3.

FIG. 5 is a side elevation view of the connector body cap of FIGS. 3 and 4.

FIG. 6 is a cross-sectional view of the connector body cap of FIGS. 3-5.

FIG. 7 is a side elevation view of one embodiment of a connector body.

FIG. 8 is front elevation view of the connector body of FIG. 7.

FIG. 9 is a perspective view of the connector body of FIGS. 7 and 8.

FIG. 10 is a perspective view of one embodiment of a saddle configured for use in connection with various spinal fixation system connectors.

FIG. 11 is a side elevation view of the saddle of FIG. 10.

FIG. 12 is a front elevation view of the saddle of FIGS. 10 and 11.

FIG. 13 is a perspective view of another embodiment of a saddle configured for use in connection with various spinal fixation system connectors.

FIG. 14 is a side elevation view of the saddle of FIG. 13.

FIG. 15 is a front elevation view of the saddle of FIGS. 13 and 14.

FIG. 16 is a perspective view of several components of an alternative embodiment of a spinal fixation system.

FIG. 17 is a perspective view of the components shown in FIG. 16 with the connector body cap removed.

### DETAILED DESCRIPTION OF PREFERRED EMBODIMENTS

In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments.

Disclosed are embodiments of spinal fixation system connectors. Some embodiments of the invention disclosed herein may be used to connect an engagement member, such as a bone screw or hook, to a spinal fixation rod. Other embodiments of the invention may be used to connect a spinal fixation rod to another connector that may, in turn, be connected to an engagement member that is engaged with a portion of a patient's vertebral column.

In one example of an embodiment according to the invention, a connector body is provided comprising a slot. The slot may be configured to allow a spinal fixation rod to extend therethrough. The connector body may also comprise two or more opposing sidewalls. In some embodiments, two opposing sidewalls may at least partially define the slot. A cap, which is configured to be connected with the connector body, may also be provided. In some embodiments, the cap may be configured to be engaged with the connector body by rotating the cap with respect to the connector body.

The cap may comprise a threaded opening configured to receive a set screw used to engage/lock the rod. The cap may also be configured to circumscribe a portion of the rod when the cap is connected with a connector body having a rod extending therethrough. The cap may also comprise two interior groove portions. The interior groove portions on the cap may be configured to receive the top portions of the two opposing sidewalls when the cap is engaged with the connector body. The two interior groove portions may comprise portions of a single circumferential groove formed in the cap or, alternatively, they may comprise separate grooves in the cap that do not form a single circumferential groove in the cap. In some embodiments having a single circumferential groove with two interior groove portions, the groove may be “open” at opposite sides of the cap such that along the open portions the groove is bounded by less than three surfaces.

In some connector embodiments, the two opposing sidewalls of the connector body, along with the two interior groove portions of the cap, may be curved. In such embodiments, the curvature of the opposing sidewalls may be substantially the same as the curvature of the interior groove portions such that the cap can be at least partially rotated with the top portions of the opposing sidewalls positioned within the interior groove portions.

One or both of the opposing sidewalls of the connector body may also comprise an exterior flange. The cap may also comprise one or more channels that are configured to receive the exterior flange(s). In some embodiments, the cap may comprise opposing sidewalls, one or both of which may have such a channel formed therein. An exterior surface of each of the exterior flanges and a surface of each of the channels may also be curved. The curvature of the exterior surfaces of the exterior flanges may also be substantially the same as the curvature of the surfaces of the channels so as to further facilitate rotatably connecting the cap with the connector body and to provide a secure connection therebetween.

A projection, such as a peg, may also be positioned adjacent to at least one of the channels to prevent over-rotation of the cap on the connector body. Accordingly, in some embodiments, the top portions of the opposing sidewalls of the connector body may be inserted into the interior groove portions in the interior of the cap, after which the cap may be rotated with respect to the connector body to engaged the flange(s) with the channel(s). Further rotation of the cap is then prevented when the projection(s) abut the exterior flange(s). In other embodiments, the top portions of the opposing sidewalls of the connector body may be engaged with the interior groove portions of the cap simultaneously with the engagement of the flange(s) with the channel(s).

A recess may also be positioned adjacent to at least one of the flanges so as to receive the projection to thereby engage the cap with the connector body at a fixed rotational position. The recess may be sized and/or shaped to correspond with the size and/or shape of the projection so that the projection at least partially fits and locks within the recess. The interface between the recess and the projection may also be such that an audible sound—such as a “click”—is generated when the projection has been engaged with the recess, thereby signaling to the surgeon that the cap is accurately positioned on the connector body.

In embodiments that are configured for use with bone screws or other engagement members, the connector body may be configured to engage and operate in conjunction with an at least partially-spherical head. More particularly, the connector body may be configured to receive and engage the at least partially spherical head such that the bone screw can initially be positioned at any of a plurality of angles with respect to the connector body. In such embodiments, a central opening may therefore be provided to allow a bone screw or other engagement member to be inserted therethrough.

In some embodiments, this may be facilitated by providing a saddle configured to be positioned so as to simultaneously engage a bone screw and a rod. In one embodiment of such a saddle, the saddle may comprise a cutout portion configured to receive the rod therein and a seat portion configured to receive the at least partially spherical head. The saddle may also comprise an opening extending through the cutout portion and configured to allow for access to a socket on the at least partially spherical head. The socket on the head of the bone screw may be configured to engage a driving instrument to thread the bone screw into a vertebral bone.

Other embodiments of the invention may be configured for use in connection with other spinal fixation components, some of which need not be configured for direct engagement with a portion of the vertebral column. For example, in one embodiment, a spinal fixation system connector may be provided that comprises an offset connector rod extending from the connector body. Such a connector may, like the other embodiments discussed above, be configured to receive a spinal fixation rod. However, the offset connector rod, unlike the engagement members discussed above, may be engaged with another connector, which, in turn, may be connected with an engagement member, such as a bone screw. The offset connector may, in other words, be used for connecting an engagement member at a location offset from the axis of a spinal fixation rod.

Often, the offset connector rod will extend from the connector body in a direction substantially perpendicular to the direction with which the rod extends through the slot. However, of course, this need not be the case. Other embodiments are contemplated in which the offset connector rod extends at a non-perpendicular angle relative to the axis of the spinal fixation rod.

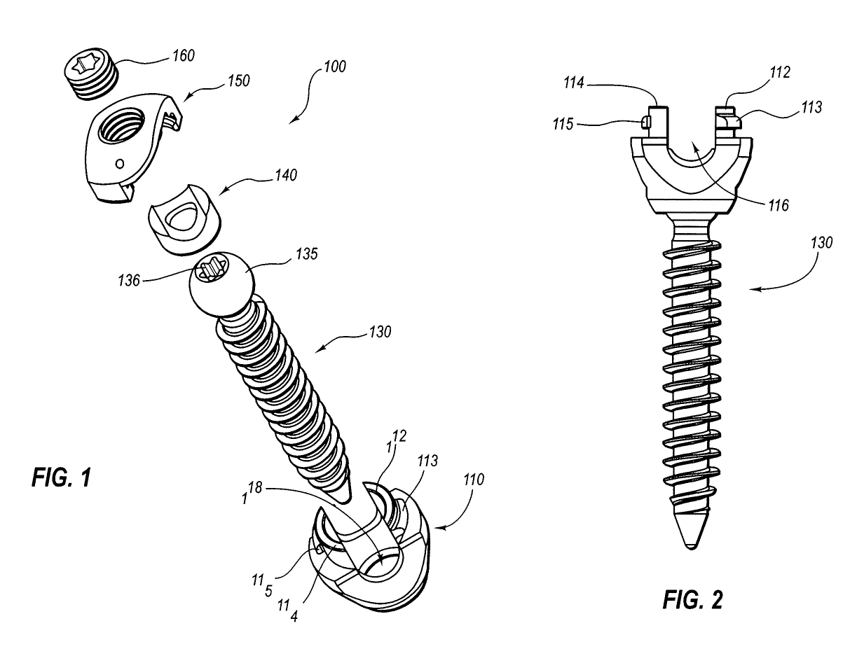

With reference to the accompanying drawings, specific examples of embodiments of the invention will now be shown and described in greater detail. FIG. 1 depicts various components of one embodiment of a spinal fixation system 100. Spinal fixation system 100 includes a connector body 110, a bone screw 130, a saddle 140, and a connector body cap 150.

Connector body 110, a side elevation view of which is shown in FIG. 2, comprises two opposing sidewalls 112 and 114, both of which are curved. In this embodiment, the curvature of sidewall 112 is the same as the curvature of sidewall 114 such that the two sidewalls are at least approximately mirror images of one another. A slot 116 is defined through the center of the connector body 110. Slot 116 is configured to allow a rod, such as a spinal fixation rod, to extend therethrough, and is partially defined by sidewalls 112 and 114. A central hole or opening 118 is also provided, which allows bone screw 130 to extend therethrough.

FIG. 2 depicts the connector body 110 and bone screw 130 after bone screw 130 has been inserted through opening 118. Note that bone screw 130 has a head 135 having a diameter which is greater than the diameter of opening 118, such that bone screw 130 cannot extend all the way through opening 118. Instead, bone screw head 135 engages the portion of connector body 110 that defines opening 118.

Opposing sidewalls 112 and 114 each comprise an exterior flange. More particularly, sidewall 112 comprises exterior flange 113 and sidewall 114 comprises exterior flange 115. The outer surface of flanges 113 and 114 are also curved. The curvature of the flanges in this embodiment is substantially the same as the curvature of the opposing sidewalls.

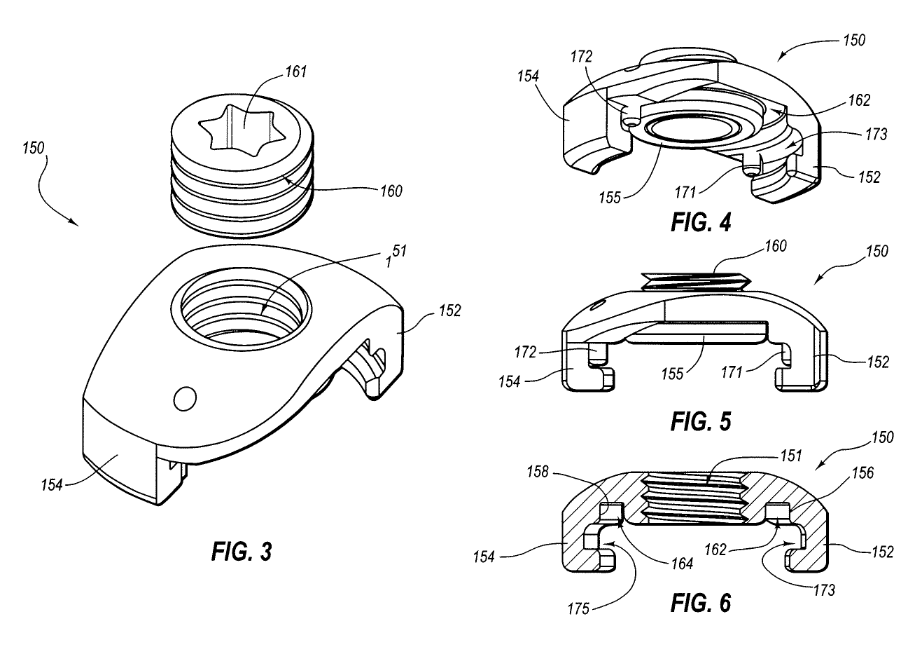

Connector body cap 150, which is shown in greater detail in FIGS. 3-6, is configured to be connected with connector body 110 and thereby circumscribe a portion of a rod (not shown). Cap 150, like connector body 110, comprises two sidewalls 152 and 154. Cap 150 also comprises two interior groove portions 162 and 164 (best seen in the cross-sectional view of FIG. 6). Interior groove portions 162 and 164 are configured to receive the top portions of the two opposing sidewalls 112 and 114 when cap 150 is engaged with connector body 110. The inside surfaces of groove portions 162 and 164 are defined by raised circular ridge 155, which defines, on one side, a single circumferential groove. The two interior groove portions 162 and 164 therefore comprise portions of a single circumferential groove formed in the cap 150. The outside surfaces of groove portions 162 and 164 are defined by curved surfaces 156 and 158. However, because surfaces 156 and 158 only extend around the perimeter of connector body cap 150 to the same extent that sidewalls 112 and 114 do, the circumferential groove defined by raised circular ridge 155 and defined in part by groove portions 162 and 164 comprises “open” portions on opposing sides that are defined only by circular ridge 155 on the inside of cap 150 and are not further defined by an outside surface.

By inserting the top portions of sidewalls 112 and 114 into groove portions 162 and 164, respectively, splaying of sidewalls 112 and 114 may be prevented, or at least the likelihood of such splaying reduced. Moreover, by substantially matching the curvature of sidewalls 112 and 114 with that of groove portions 162 and 164, rotational engagement of cap 150 with respect to connector body 110 may be facilitated. More particularly, by configuring the curvature of the opposing sidewalls to be substantially the same as the curvature of the interior groove portions, the cap can be at least partially rotated with the top portions of the opposing sidewalls positioned within the interior groove portions.

As previously mentioned, opposing sidewalls 112 and 114 each comprise an exterior flange 113 and 115, respectively. In the depicted embodiment, the cap 150 also comprises opposing sidewalls 152 and 154, as also previously mentioned. The opposing cap sidewalls 152 and 154 each comprise an inside channel 173 and 175, respectively, each of which is configured to receive an exterior flange from the connector body 110. More particularly, channel 173 is configured to receive flange 113 from connector body 110 and channel 175 is configured to receive flange 115 from connector body 110. Thus, as cap 150 is rotated with sidewalls 112 and 114 engaging groove portions 162 and 164, flanges 113 and 115 are also rotated into engagement with channels 173 and 175, respectively. In the depicted embodiment, flanges 113 and 115 do not extend along the entire length of sidewalls 112 and 114. As such, cap 150 can be lowered onto connector body 110 with parts (corresponding with the length of sidewalls 112 and 114 lacking a flange) of sidewalls 112 and 114 positioned within groove portions 162 and 164, after which cap 150 can be rotated to further engage cap 150 with connector body 110 by engaging flanges 113 and 115 with channels 173 and 175. When cap 150 has been rotated into this position, it cannot be pulled off of connector body 110 without further rotation of cap 150.

In some embodiments, one or more projections may be provided on the cap. For example, cap 150 includes projections 171 and 172. Projections 171 and 172 comprise pegs, although other types of projections—such as rails, pins, teeth, and the like—may alternatively be used. The projection(s) may be positioned adjacent to at least one of the channels to prevent over-rotation of the cap on the connector body. Thus, for example, peg 171 is positioned adjacent to channel 173 and peg 172 is positioned adjacent to channel 175, as best shown in FIGS. 4 and 5. Pegs 171 and 172 contact flanges 113 and 115 once cap 150 has been fully rotated into position on connector body 110 so as to prevent further rotation of cap 150 in the same direction. Any of the aforementioned projections, including pegs 171 and 172, are examples of means for preventing over-rotation of a connector body cap with respect to a connector body.

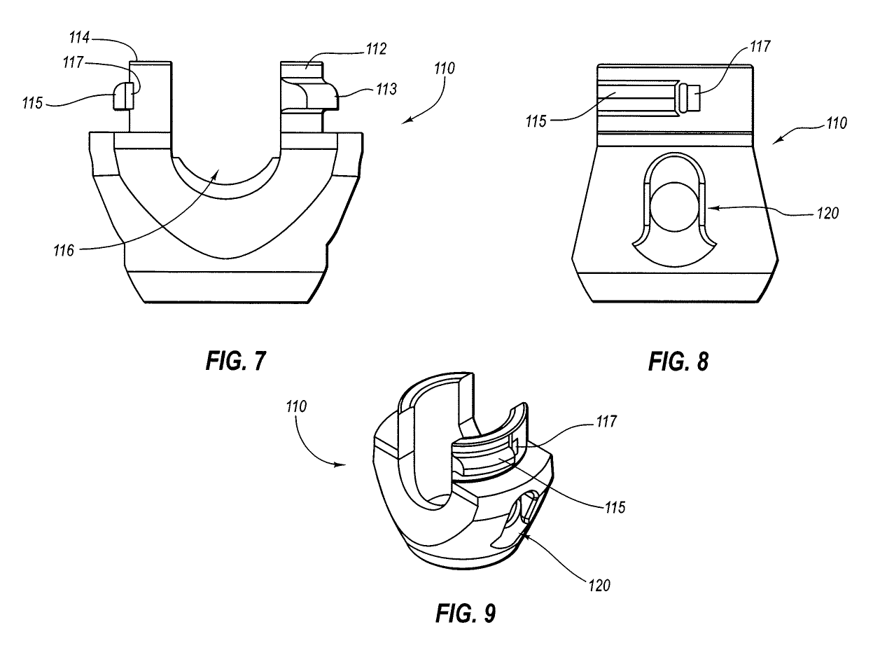

In some embodiments, a recess may be positioned adjacent to one or more of the flanges on the connector body. This recess (or recesses) may be configured to receive a projection on the connector body cap to engage the cap with the connector body at a fixed rotational position. In some embodiments, the recess and the projection may be configured such that an audible sound—such as a “click”—is generated when the projection is received within and engages the recess, thereby signaling to the surgeon that the cap has been fully rotated into place on the connector body. Thus, for example, connector body 110 comprises recess 117, as shown in FIGS. 7 and 9. Recess 117 has a dimension (width in this case) that approximately matches that of peg 172. When cap 150 has been fully rotated and engaged with connector body 110, peg 172 contacts flange 115 and thereby prevents cap 150 from rotating further.

Likewise, due to the “snap-fit” between peg 172 and recess 117, rotation of cap 150 in the opposite direction is made more difficult. As such, engagement of cap 150 with connector body 110 in a particular fixed rotational orientation, and maintenance of such engagement, is facilitated. Of course, the projection/recess engagement may be such that a moderate to high amount of force can dislodge the projection from the recess. In addition, recesses may be provided adjacent to both flanges if desired. Recess 117, along with projection 171, comprise one example of a means for selectively maintaining a fixed rotational orientation between a connector body cap and a connector body.

As best seen in FIG. 3, the connector body cap 150 also comprises a threaded opening 151 configured to receive a set screw 160. Set screw 160 may comprise a feature 161 for receiving a screwdriver or another similar driving instrument. The set screw 160 may be used to engage a spinal fixation rod extending through slot 116. In some embodiments, tightening of set screw 160 may also result in fixation of a bone screw with respect to the connector body 110. Thus, whereas the connector body 110 may be configured to initially receive and engage a head of bone screw 130 such that the bone screw 130 can be positioned at any of a plurality of angles with respect to the connector body 110, tightening of set screw 160 may clamp down on the rod, which, in turn, may engage and clamp down on the top of bone screw head 135 (in some embodiments via saddle 140) to lock the relative angle between bone screw 130 and connector body 110.

Connector body 110 may also include one or more staking windows 120 in some embodiments, as best shown in FIG. 8. Staking windows 120 may comprise a thinned or recessed portion in connector body 110 or, alternatively, may comprise an opening extending all the way through one wall of the connector body. As described in greater detail below, staking windows 120 may also be configured to interact with saddle 140.

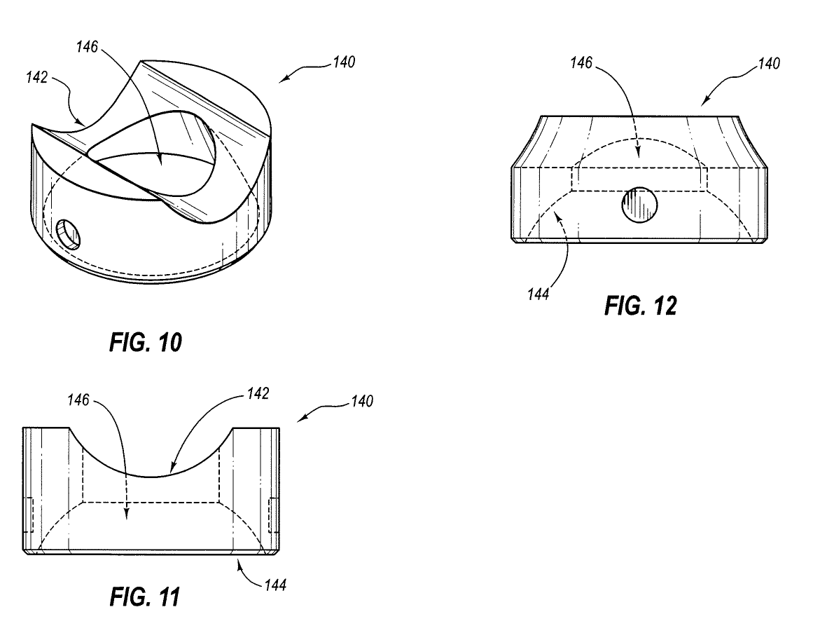

One embodiment of saddle 140 is shown in greater detail in FIGS. 10-12. Saddle 140 is positioned and configured to simultaneously engage bone screw 130 and a spinal fixation rod (not shown). As demonstrated by these drawings, saddle 140 comprises a cutout portion 142 configured to receive the rod therein. Opposite cutout portion 142, a seat portion 144 is provided, which is configured to receive the at least partially spherical head 135 of bone screw 130. Seat portion 144 may be frusto-spherical. As such, the at least partially spherical head 135 of bone screw 130 may be able to pivot while seated within seat portion 144. In other embodiments, seat portion 144 may be frusto-conical.

Saddle 140 may also comprise an opening 146 configured to allow for access to a socket 136 on the at least partially spherical head 135 of bone screw 130. Socket 136 may be configured to engage a driving instrument to thread the bone screw 130 into a vertebral bone.

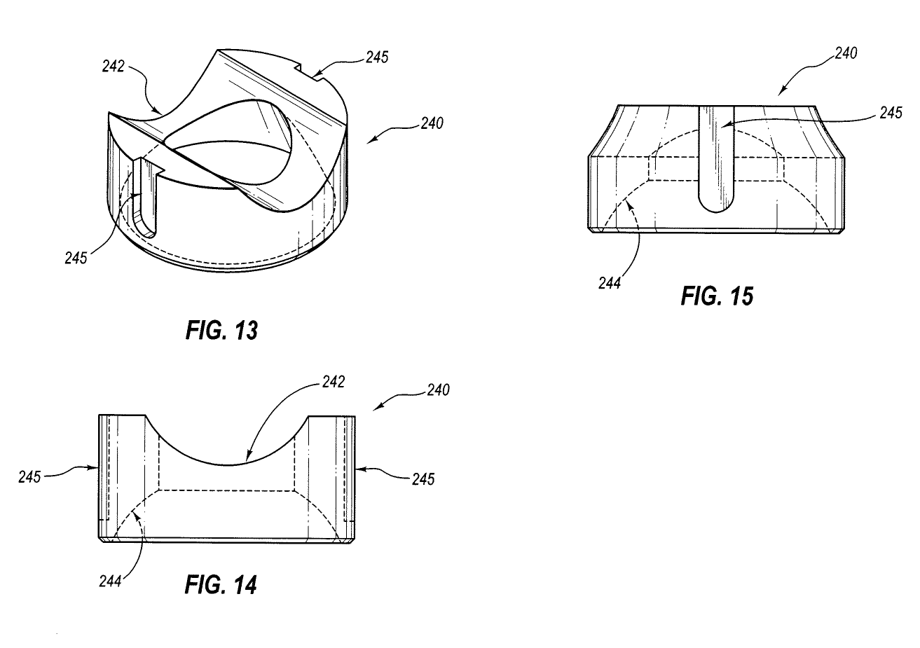

An alternative saddle 240 is shown in FIGS. 13-15. Like saddle 140, saddle 240 comprises a cutout portion 242 configured to receive a spinal fixation rod therein. Opposite cutout portion 242, a seat portion 244 is provided, which is configured to receive a bone screw head. However, unlike saddle 140, saddle 240 includes staking slots 245. Staking slots 245 may each comprise a thinned or cutout portion of one wall of saddle 240. Staking slots 245 may be aligned with staking windows 120 of connector body 110. In other words, a portion of the wall of connector body 110 which defines staking window 120 may be staked into staking slot 245 to thereby retain saddle 240 within connector body 110.

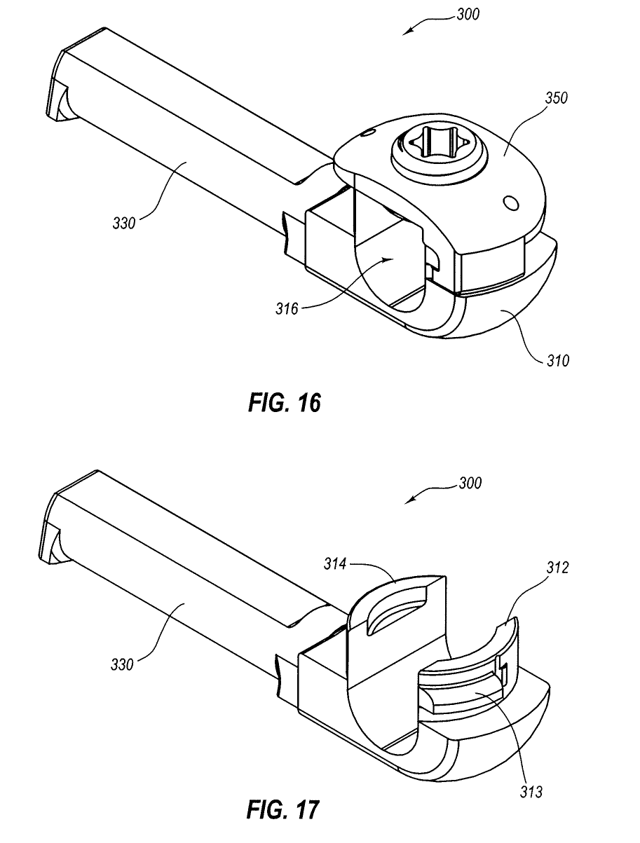

An alternative embodiment of a spinal fixation system 300 is shown in FIGS. 16 and 17. Spinal fixation system 300 comprises connector body 310, which is configured to be rotatably engaged with cap 350. Spinal fixation system 300 also comprises an offset connector rod 330 extending from the connector body 310. Offset connector rod 330 extends from connector body 310 in a direction substantially perpendicular to the direction with which a spinal fixation rod extends through the slot 316 defined through the center of connector body 310. Of course, other embodiments are contemplated in which this is not the case.

The cap/connector body interface for the alternative embodiment shown in FIGS. 16 and 17 may include features similar to those provided with connector body 110 and cap 150. For example, FIG. 17 depicts connector body 310 with opposing sidewalls 312 and 314. One or more exterior flanges 313 may also be provided for engaging features on the cap 350 (not shown).

Spinal fixation system 300 allows for offset connection of a spinal fixation engagement member, such as a bone screw. In other words, offset connector rod 330 may be inserted into a connector body, such as connector body 110, of another spinal fixation component. Thus, connector body 310 may, like connector body 110, be configured to receive a spinal fixation rod. However, offset connector rod 330 may be engaged with another connector body, which, in turn, may be connected with an engagement member, such as a bone screw. Offset connector 330 may thereby be used for connecting an engagement member, such as a bone screw, at a location offset from the axis of a spinal fixation rod.

The above description fully discloses the invention including preferred embodiments thereof. Without further elaboration, it is believed that one skilled in the art can use the preceding description to utilize the invention to its fullest extent. Therefore the examples and embodiments disclosed herein are to be construed as merely illustrative and not a limitation of the scope of the present invention in any way.

It will be obvious to those having skill in the art that many changes may be made to the details of the above-described embodiments without departing from the underlying principles of the invention. The scope of the present invention should, therefore, be determined only by the following claims.

1. A spinal fixation system connector, comprising:
a connector body comprising a slot configured to allow a rod to extend therethrough, wherein the connector body comprises at least two opposing sidewalls;
a cap configured to be connected with the connector body and thereby circumscribe a portion of the rod, wherein the cap comprises at least two interior groove portions, and wherein the two interior groove portions are configured to receive the top portions of the two opposing sidewalls when the cap is engaged with the connector body.

2. The spinal fixation system connector of claim 1, wherein the two interior groove portions comprise portions of a single circumferential groove formed in the cap.

3. The spinal fixation system connector of claim 2, wherein the single circumferential groove is open at opposite sides of the cap.

4. The spinal fixation system connector of claim 1, wherein the two opposing sidewalls are curved, wherein the two interior groove portions are curved, and wherein the curvature of the opposing sidewalls is substantially the same as the curvature of the interior groove portions such that the cap can be at least partially rotated with the top portions of the opposing sidewalls positioned within the interior groove portions.

5. The spinal fixation system connector of claim 1, wherein the opposing sidewalls each comprise an exterior flange, wherein the cap comprises opposing sidewalls, and wherein the opposing cap sidewalls each comprise a channel that is configured to receive an exterior flange.

6. The spinal fixation system connector of claim 5, further comprising a projection positioned adjacent to at least one of the channels and configured to prevent over-rotation of the cap on the connector body.

7. The spinal fixation system connector of claim 6, wherein the projection comprises a peg.

8. The spinal fixation system connector of claim 6, further comprising a recess positioned adjacent to at least one of the flanges, wherein the recess is configured to receive the projection to engage the cap with the connector body at a fixed rotational position.

9. The spinal fixation system connector of claim 1, wherein the cap comprises a threaded opening configured to receive a set screw.

10. The spinal fixation system connector of claim 1, wherein the connector body is further configured to be coupled with a bone screw.

11. The spinal fixation system connector of claim 10, wherein the connector body is configured to receive and engage an at least partially spherical head of the bone screw such that the bone screw can initially be positioned at any of a plurality of angles with respect to the connector body.

12. The spinal fixation system connector of claim 11, wherein the connector body further comprises a saddle positioned and configured to simultaneously engage the bone screw and the rod.

13. The spinal fixation system connector of claim 12, wherein the saddle comprises:
a cutout portion configured to receive the rod therein; and
a seat portion configured to receive the at least partially spherical head.

14. The spinal fixation system connector of claim 13, wherein the saddle comprises an opening configured to allow for access to a socket on the at least partially spherical head and wherein the socket is configured to engage a driving instrument to thread the bone screw into a vertebral bone.

15. The spinal fixation system connector of claim 1, further comprising an offset connector rod extending from the connector body.

16. The spinal fixation system connector of claim 15, wherein the offset connector rod extends from the connector body in a direction substantially perpendicular to the direction with which the rod extends through the slot.

17. A system for connecting a spinal fixation rod to a vertebral column, the system comprising:
an engagement member configured to be engaged with a portion of the vertebral column;
a connector body configured to be connected with the engagement member, wherein the connector body comprises a slot configured to allow the rod to extend therethrough, and wherein the connector body comprises at least two opposing sidewalls; and
a cap configured to be engaged with the connector body by rotating the cap with respect to the connector body, wherein the cap comprises at least two interior groove portions, and wherein the two interior groove portions are configured to receive the top portions of the two opposing sidewalls when the cap is engaged with the connector body.

18. The system of claim 17, wherein the at least two opposing sidewalls at least partially define the slot.

19. The system of claim 17, wherein the engagement member comprises a bone screw.

20. The system of claim 17, wherein the two opposing sidewalls are curved, wherein the two interior groove portions are curved, and wherein the curvature of the opposing sidewalls is substantially the same as the curvature of the interior groove portions such that the cap can be at least partially rotated with the top portions of the opposing sidewalls positioned within the interior groove portions.

21. The system of claim 20, wherein the connector body further comprises at least two exterior flanges, wherein the cap comprises at least two channels that are configured to receive the exterior flanges.

22. The system of claim 21, wherein an exterior surface of each of the exterior flanges is curved, wherein a surface of each of the channels is curved, and wherein the curvature of the exterior surfaces of the exterior flanges is substantially the same as the curvature of the surfaces of the channels.

23. The system of claim 22, further comprising means for preventing over-rotation of the cap with respect to the connector body.

24. The system of claim 23, wherein the means for preventing over-rotation of the cap with respect to the connector body comprises a projection positioned adjacent to at least one of the channels.

25. The system of claim 23, further comprising means for selectively maintaining a fixed rotational orientation between the cap and the connector body.

26. The system of claim 25, wherein the means for selectively maintaining a fixed rotational orientation between the cap and the connector body is configured to engage the means for preventing over-rotation of the cap with respect to the connector body.

27. The system of claim 26, wherein the means for selectively maintaining a fixed rotational orientation between the cap and the connector body comprises a recess configured to engage a projection positioned adjacent to at least one of the channels.

28. The system of claim 17, further comprising a saddle positioned and configured to simultaneously engage the engagement member and the rod.

29. The system of claim 28, wherein the saddle comprises:
a cutout portion configured to receive the rod therein; and
a seat portion configured to receive an at least partially spherical head of the engagement member.

30. The system of claim 28, wherein the engagement member comprises a bone screw having a head, wherein the head comprises a socket configured to engage a driving instrument to thread the bone screw into a vertebral bone, and wherein the saddle comprises an opening configured to allow for access to the socket.

31. A system for connecting a spinal fixation rod to a vertebral column, the system comprising:
a bone screw having an at least partially spherical head, wherein the head comprises a socket configured to engage a driving instrument;
a connector body connected with the bone screw, wherein the connector body comprises at least two curved opposing sidewalls at least partially defining a slot, wherein the slot is configured to allow the rod to extend therethrough, and wherein the opposing sidewalls each comprise an exterior flange;
a cap configured to be engaged with the connector body by rotating the cap with respect to the connector body, wherein the cap comprises at least two curved interior groove portions configured to receive the top portions of the two curved opposing sidewalls when the cap is engaged with the connector body, wherein the curvature of the opposing sidewalls is substantially the same as the curvature of the interior groove portions such that the cap can be at least partially rotated with the top portions of the opposing sidewalls positioned within the interior groove portions, wherein the cap comprises at least two channels that are configured to receive the exterior flanges, wherein the cap comprises at least one projection positioned adjacent to at least one of the channels and configured to prevent over-rotation of the cap on the connector body, wherein the connector body comprises a recess positioned adjacent to at least one of the flanges, wherein the recess is configured to receive the projection to engage the cap with the connector body at a fixed rotational position, and wherein the cap comprises a threaded opening configured to receive a set screw; and
a saddle positioned and configured to simultaneously engage the head and the rod, wherein the saddle comprises a cutout portion configured to receive the rod therein and a seat portion configured to receive the at least partially spherical head, and wherein the saddle comprises an opening configured to allow for access to the socket.
